(19) United States
(12) Patent Application Publication
(10) Pub. No.: US 2026/0073534 A1
(43) Pub. Date: Mar. 12, 2026

(54) Motion Detection in Image-Guided Thermal Therapy Using Trained Machine-Learning Model

(71) Applicant: Profound Medical Inc., Mississauga (CA)

(72) Inventor: Elyas Shaswary, Milton (CA)

(21) Appl. No.: 19/326,964

(22) Filed: Sep. 12, 2025

Related U.S. Application Data
(60) Provisional application No. 63/693,799, filed on Sep. 12, 2024.

(51) Int. Cl. G06T 7/246; A61B 34/20; G06T 7/00; G06T 7/50

(52) U.S. Cl. CPC G06T 7/248; A61B 34/20; G06T 7/0012; G06T 7/50; A61B 2034/2051; G06T 2207/10088

(57) ABSTRACT

A reference shape of each target object is determined from one or more of reference MR images using one or more trained machine-learning (ML) models, each reference MR image captured at a respective spatial location in the target volume. A subsequent shape of each target object is determined from one or more of subsequent MR images using the trained ML model(s), each reference MR image captured at the same respective spatial location in the target volume as a corresponding reference MR image. A respective movement of each target object is calculated based, at least in part, on a comparison of each subsequent shape for each target object to a corresponding reference shape for a corresponding target object at the same respective spatial location in the target volume. When the respective movement is greater than a predetermined threshold, a movement notification is produced.

### CROSS-REFERENCE TO RELATED APPLICATIONS

This application claims priority to U.S. Provisional Application No. 63/693,799, filed on Sep. 12, 2024, titled “Motion Detection in Image-Guided Thermal Therapy Using Trained Machine-Learning Model,” which is hereby incorporated by reference.

### TECHNICAL FIELD

This application relates generally to thermal therapy.

### BACKGROUND

Image-guided thermal therapy is used to treat a variety of conditions and diseases including cancer. During thermal therapy, magnetic resonance images are captured of the target volume to monitor the temperature using MRI thermometry. MRI thermometry relies on variations in the relative phase of current MR images compared to baseline MR images and thus is highly sensitive to patient movement. Small movements of the patient are difficult to detect and can cause the temperature to shift. When the measured temperatures are depressed, continuing thermal therapy can cause increase the temperature of nearby healthy anatomical features leading to damage while temperature safety limits appear to be met. When the measured temperatures are raised, continuing thermal therapy can cause the temperature safety limits to be reached prematurely, causing the procedure to be temporarily stopped while the target volume cools.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages, and novel features of the disclosure will be set forth in the following detailed description of the disclosure when considered in conjunction with the drawings, which are intended to illustrate, not limit, the invention.

An aspect of the invention is directed to a computer configured to monitor motion during a medical procedure, comprising: one or more processors; and non-volatile computer-readable memory operably coupled to the one or more processors, the non-volatile computer-readable memory storing computer-readable instructions that, when executed by the one or more processors, cause the one or more processors to: receive reference magnetic resonance (MR) images of a target volume of a mammal, the target volume including one or more target objects, the reference MR images captured over a first time period; determine a reference shape of each target object from one or more of the reference MR images using one or more trained machine-learning (ML) models running on the computer, each reference MR image captured at a respective spatial location in the target volume; receive subsequent MR images of the target volume, the subsequent MR images captured over a second time period that occurs after the first time period; determine a subsequent shape of each target object from one or more of the subsequent MR images using the one or more trained ML models, each subsequent MR image captured at the same respective spatial location in the target volume as a corresponding reference MR image; compare each subsequent shape for each target object to a corresponding reference shape for a corresponding target object, wherein a comparison of a given subsequent shape and a given reference shape is performed using a corresponding subsequent MR image and a corresponding reference MR image that were captured at the same respective spatial location in the target volume; calculate a respective movement of each target object based, at least in part, on the comparison; and when the respective movement is greater than a predetermined threshold, produce a movement notification.

In one or more embodiments, the one or more target objects includes one or more target anatomical features of the mammal. In one or more embodiments, the one or more target anatomical features includes a prostate.

In one or more embodiments, the one or more target objects includes one or more medical devices. In one or more embodiments, the one or more medical devices includes a thermal therapy applicator and/or an endorectal cooling device. In one or more embodiments, the computer-readable instructions, when executed by the one or more processors, further cause the one or more processors to cause the thermal therapy applicator to start a thermal therapy procedure after determining the reference shape of each target object. In one or more embodiments, the computer-readable instructions, when executed by the one or more processors, further cause the one or more processors to: compare the reference shape of the endorectal cooling device with a known shape of the of the endorectal cooling device; and produce a physical obstruction notification when the reference shape is different than the known shape.

In one or more embodiments, the computer-readable instructions, when executed by the one or more processors, further cause the one or more processors to: calculate a reference centroid of each reference shape; calculate a subsequent centroid of each subsequent shape; determine a respective distance between a position of each subsequent centroid for each subsequent shape to a position of a corresponding reference centroid for the corresponding reference shape; and calculate the respective movement of each target object based, at least in part, on the respective distance.

In one or more embodiments, the first time period occurs before a start of the medical procedure and the second time period occurs during the medical procedure.

Another aspect of the invention is directed to a method for controlling a delivery of thermal therapy, comprising: inserting a thermal therapy applicator into a mammal; capturing first magnetic resonance (MR) images, with an MR imaging system, of the mammal at a first time, the first MR images representing first cross-sectional images of the mammal including an inserted thermal therapy applicator, the first cross-sectional images at respective spatial locations in the mammal; segmenting the first MR images with a trained machine-learning (ML) model running on the computer, the trained ML model having been trained with reference segmented MR images that include a reference thermal therapy applicator; determining, with the computer, a respective first shape and/or a respective first position of the inserted thermal therapy applicator at each spatial location; applying thermal therapy, with the inserted thermal therapy applicator, to a target volume in the mammal; and while applying the thermal therapy: a. capturing second MR images, with the MR imaging system, of the mammal at a second time, the second MR images representing second cross-sectional images of the mammal and the inserted thermal therapy applicator, the second cross-sectional images at the respective spatial locations; b. segmenting the second MR images with the trained ML model; c. determining, with the computer, a respective second shape and/or a respective second position of the inserted thermal therapy applicator at each spatial location; d. calculating, with the computer, a displacement of the inserted thermal therapy applicator at each spatial location by comparing the respective first and second shapes and/or the respective first and second positions of the inserted thermal therapy applicator at each spatial location; and c. producing a notification, with the computer, when the displacement of the inserted thermal therapy applicator is greater than a predetermined threshold value.

In one or more embodiments, the method further comprises preprocessing the first MR images, wherein segmenting the first MR images comprises segmenting first preprocessed MR images; and preprocessing the second MR images, wherein segmenting the second MR images comprises segmenting second preprocessed MR images. In one or more embodiments, preprocessing the first MR images includes: extracting magnitude data for each first MR image; and normalizing the magnitude data for each first MR image; and preprocessing the second MR images includes: extracting magnitude data for each second MR image; and normalizing the magnitude data for each second MR image.

In one or more embodiments, the method further comprises displaying, on a display screen in communication with the computer, an overlay of (a) one or more of the second MR images and (b) the respective second shape and/or the respective second position of the inserted thermal therapy applicator corresponding to the one or more of the second MR images. In one or more embodiments, the method further comprises determining, with the computer, a respective first centroid of the respective first shape; and determining, with the computer, a respective second centroid of the respective second shape, wherein the respective displacement is calculated using the respective first and second centroids corresponding to the respective spatial location.

In one or more embodiments, the method further comprises repeating steps a-c in a loop while applying the thermal therapy.

Another aspect of the invention is directed to a system for controlling a delivery of thermal therapy, comprising: a magnetic resonance (MR) imaging system; a computer in communication with the MR imaging system, the computer including: one or more processors; and non-volatile computer-readable memory operably coupled to the one or more processors, the non-volatile computer-readable memory storing computer-readable instructions that, when executed by the one or more processors, cause the one or more processors to: receive first MR images of a mammal at a first time, the first MR images representing first cross-sectional images of the mammal including an inserted thermal therapy applicator, the first cross-sectional images at respective spatial locations in the mammal; segment the first MR images with a trained machine-learning (ML) model running on the computer, the trained ML model having been trained with reference segmented MR images that include a reference thermal therapy applicator; determine a respective first shape and/or a respective first position of the inserted thermal therapy applicator at each spatial location; while the thermal therapy is applied with the inserted thermal therapy applicator: a. receive second MR images of the mammal at a second time, the second MR images representing second cross-sectional images of the mammal and the inserted thermal therapy applicator, the second cross-sectional images at the respective spatial locations; b. segment the second MR images with the trained ML model; c. determine a respective second shape and/or a respective second position of the inserted thermal therapy applicator at each spatial location; d. calculate a displacement of the inserted thermal therapy applicator at each spatial location by comparing the respective first and second shapes and/or the respective first and second positions of the inserted thermal therapy applicator at each spatial location; and c. produce a notification when the displacement of the inserted thermal therapy applicator is greater than a predetermined threshold value.

In one or more embodiments, the computer-readable instructions, when executed by the one or more processors, further cause the one or more processors to display, on a display screen in communication with the computer, an overlay of (a) one or more of the second MR images and (b) the respective second shape and/or the respective second position of the inserted thermal therapy applicator corresponding to the one or more of the second MR images. In one or more embodiments, the computer-readable instructions, when executed by the one or more processors, further cause the one or more processors to: determine a respective first centroid of the respective first shape; and determine a respective second centroid of the respective second shape, wherein the respective displacement is calculated using the respective first and second centroids corresponding to the respective spatial location.

In one or more embodiments, the computer-readable instructions, when executed by the one or more processors, further cause the one or more processors to repeat steps a-e in a loop the thermal therapy is applied with the inserted thermal therapy applicator. In one or more embodiments, the computer-readable instructions, when executed by the one or more processors, further cause the one or more processors to: preprocess the first MR images, wherein segmenting the first MR images comprises segmenting first preprocessed MR images; and preprocess the second MR images, wherein segmenting the second MR images comprises segmenting second preprocessed MR images.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 is a diagram of a medical system according to one or more embodiments.

FIG. 2 is a simplified anatomical view of an ultrasound applicator and an endorectal cooling device according to one or more embodiments.

FIG. 3 is a flow chart of a method for controlling delivery of thermal therapy according to one or more embodiments.

FIG. 4 shows an example set of first magnetic resonance (MR) images 400 according to one or more embodiments.

FIG. 5 shows an example shape of each of the medical device(s) for each first MR image.

FIG. 6 shows an example centroid of each of the medical device(s) for each first MR image.

FIGS. 7A and 7B are simplified diagrams of first centroids of an ultrasound applicator (UA) and an endorectal cooling device (ECD) determined from a first MR image corresponding to a first spatial location and second centroids of the UA and ECD determined from a second MR image corresponding to the same first spatial location.

FIGS. 8A and 8B are simplified diagram of first shapes of a UA and an ECD determined from a first MR image corresponding to a first spatial location and second shapes of the UA and ECD determined from a second MR image corresponding to the same first spatial location.

FIG. 9 is a flow chart of a computer-implemented method for determining a respective shape and/or a respective position of a target object in an MR image according to one or more embodiments.

FIG. 10 is a flow chart of a computer-implemented method for detecting a physical obstruction on a medical device using an MR image according to one or more embodiments.

FIG. 11 is a flow chart of a method for controlling delivery of thermal therapy according to one or more embodiments.

FIG. 12 shows an example shape of each target anatomical feature(s) for each first MR image.

FIG. 13 shows an example centroid(s) of each target anatomical feature(s) for each first MR image.

FIG. 14 is a simplified diagram of first centroids of the prostate and the rectum determined from a first MR image corresponding to a first spatial location, and second centroids of the prostate and the rectum determined from a second MR image corresponding to the same first spatial location.

FIG. 15 is a simplified diagram of first shapes of the prostate and the rectum determined from a first MR image corresponding to a first spatial location and second shapes of the prostate and the rectum determined from a second MR image corresponding to the same first spatial location.

FIG. 16 is a flow chart of a method for controlling delivery of a medical procedure, such as thermal therapy, according to one or more embodiments.

FIG. 17 is a block diagram of a system according to one or more embodiments.

### DETAILED DESCRIPTION

A reference shape and/or a reference position of one or more target objects is/are determined, using reference magnetic resonance (MR) images, before a medical procedure (e.g. thermal therapy) begins. After the medical procedure begins, a subsequent shape and/or a subsequent position of the one or more target objects is/are determined using subsequent MR images. The reference shape and the subsequent shape of each target object can be compared to determine a movement of a respective target object. Additionally or alternatively, the reference position and the subsequent position of each target object can be compared to determine a movement of a respective target object. A movement warning can be produced when the detected movement is greater than a predetermined threshold.

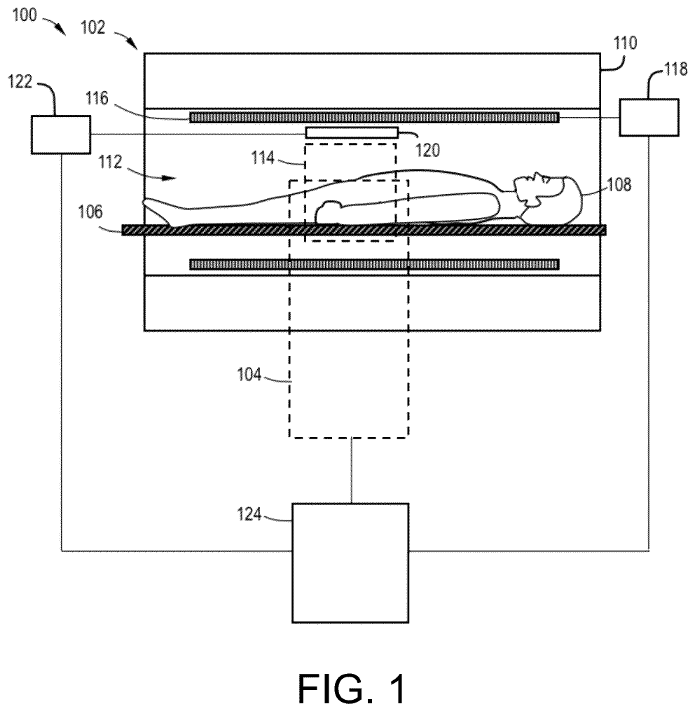

FIG. 1 is a diagram of a medical system 100 in which at least some of the apparatus, systems, and/or methods disclosed herein are employed, in accordance with at least some embodiments. The system 100 includes a patient support 106 (on which a patient 108 is shown), a magnetic resonance imaging (MRI) system 102 and an image-guided energy delivery system 104.

The magnetic resonance system 102 includes a magnet 110 disposed about an opening 112, an imaging zone 114 in which the magnetic field is strong and uniform enough to perform MRI, a set of magnetic field gradient coils 116 to change the magnetic field rapidly to enable the spatial coding of MRI signals, a magnetic field gradient coil power supply 118 that supplies current to the magnetic field gradient coils 116 and is controlled as a function of time, a transmit/receive coil 120 (also known as a “body” coil) to manipulate the orientations of magnetic spins within the imaging zone 114, a radio frequency transceiver 122 connected to the transmit/receive coil 120, and a computer 124, which performs tasks (by executing instructions and/or otherwise) to facilitate operation of the MRI system 102 and is coupled to the radio frequency transceiver 122, the magnetic field gradient coil power supply 118, and the image-guided energy delivery system 104. The image-guided energy delivery system 104 includes a therapeutic applicator, such as an ultrasound applicator, to perform image-guided therapy (e.g., thermal therapy) to treat a treatment volume in the patient 108.

The computer 124 can include more than one computer in some embodiments, at least one of which can be dedicated to the MRI system 102. In at least some embodiments, the computer 124 and/or one or more other computing devices (not shown) in and/or coupled to the system 100 may also perform one or more tasks (by executing instructions and/or otherwise) such as to control the driving or operating frequency of the ultrasound elements in the therapeutic applicator, such as at the center frequency (f₀) and/or at a higher harmonic (3f₀) of the center frequency.

One or more of the computers, including computer 124, can include a treatment plan for and/or program instructions for determining a treatment plan (e.g., in real time) for the patient 108 that includes the target treatment volume and the desired or minimal energy (e.g., thermal) dose for the target treatment volume. The treatment plan can also include the desired operating or driving frequency of the ultrasound elements, such as f₀ and/or 3f₀. The computer(s) can use images from the MRI system 102 to image guide the rotational position and insertion-retraction position of the therapeutic applicator. In some embodiments, one or more dedicated computers control the image-guided energy delivery system 104. Some or all of the foregoing computers can be in communication with one another (e.g., over a local area network, a wide area network, a cellular network, a WiFi network, or other network), for example through a software-controlled link to a communication network.

In some embodiments, the treatment plan includes a set of initial parameters for driving each ultrasound element such as its initial frequency, initial phase, and initial amplitude. These parameters can be updated in real time based on the measured temperature of the target volume, for example as determined by MR thermometry.

In other embodiments, the image-guided energy delivery system 104 can be guided with another imaging device, such as an ultrasound imaging device. In other embodiments, the image-guided energy delivery system 104 can be used without an imaging device in which case the image-guided energy delivery system 104 is an energy delivery system 104.

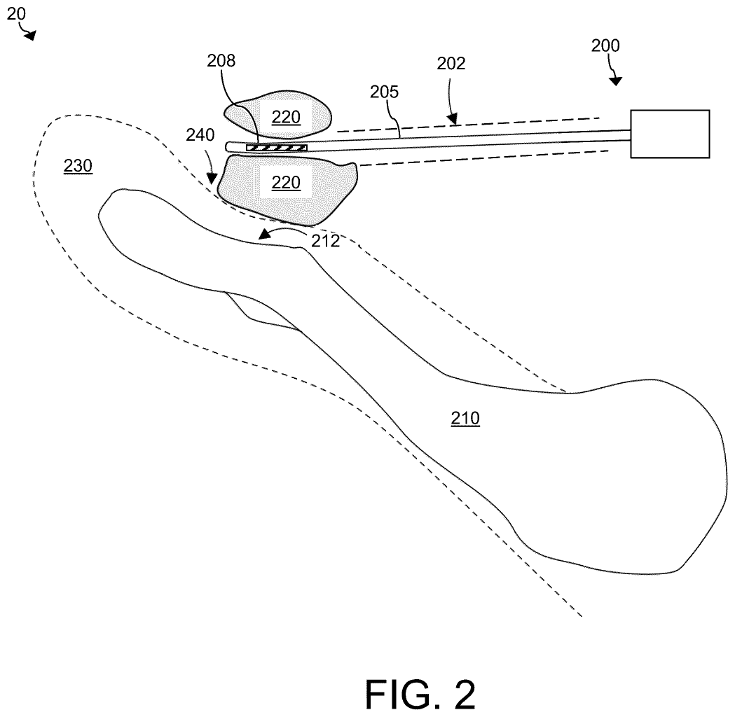

FIG. 2 is a simplified anatomical view of an ultrasound applicator (UA) 200 and an endorectal cooling device (ECD) 210 that have been inserted into a patient (e.g., a human or another mammal) 20 during a medical procedure. A shaft 205 at the distal end of the UA 200 is inserted through the urethra 202 and between the upper and lower portions of prostate 220. The UA 200 can include one or more ultrasound transducers 208, such as an ultrasound transducer array, that can produce ultrasound energy to heat the prostate 220. The image-guided energy delivery system 104 (FIG. 1) can comprise the UA 200.

The ECD 210 is inserted into the rectum 230 to cool the rectum 230 and/or a rectal wall 240 proximal to the prostate 220. The ECD 210 can include a cooling surface 212 that is shaped to conform to the shape of the rectal wall 240. A cooling fluid can circulate inside the ECD 210 to cool the ECD 210 and the cooling surface 212. For purpose of this illustration and for clarity, the distance between the ECD's cooling surface 212 and the rectal wall 240 is illustrated as larger than it is in practice.

During thermal therapy, MR images of the prostate 220, the rectum 230, the UA 200, and/or the ECD 210 are taken by a magnetic resonance system 102. The MR images can be used to monitor the temperature (e.g., through MRI thermometry) of the prostate 220 and the rectum 230 during thermal therapy and/or to determine/monitor the position(s) of the UA 200 and/or of the ECD 210 prior to and during thermal therapy. Before thermal therapy begins, the MR images can be used to position the UA 200 and/or the ECD 210 at a respective target location, for example relative to the patient anatomy. For example, the ultrasound transducer(s) 208 can be aligned with a target volume of the prostate 220 and/or the ECD 210 can be aligned with a rectal wall 240. After the UA 200 and/or the ECD 210 is/are positioned at a respective target location, the MR images can be used to monitored its/their respective position(s) during thermal therapy to determine whether the UA 200 and/or the ECD 210 has/have moved relative to their initial target location(s).

Detecting movement of the UA 200 and/or of the ECD 210 can be used (e.g., as a proxy) to determine whether the patient has moved. A warning can be automatically generated when movement of the UA 200 and/or the ECD 210 is detected. Additionally or alternatively, the thermal therapy procedure can be automatically stopped when movement of the UA 200 and/or the ECD 210 is/are detected.

In one or more embodiments, the UA 200 can be the same as the UA disclosed in U.S. Pat. No. 9,707,413, titled “Controllable Rotating Ultrasound Therapy Applicator,” which is hereby incorporated by reference. In one or more embodiments, the ECD 210 can be the same as the ECD disclosed in U.S. Pat. No. 11,596,544, titled “Gas Bubble Removal For Endorectal Cooling Devices,” and/or in U.S. Pat. No. 10,231,865, titled “Endocavity Temperature Control Device,” which are hereby incorporated by reference.

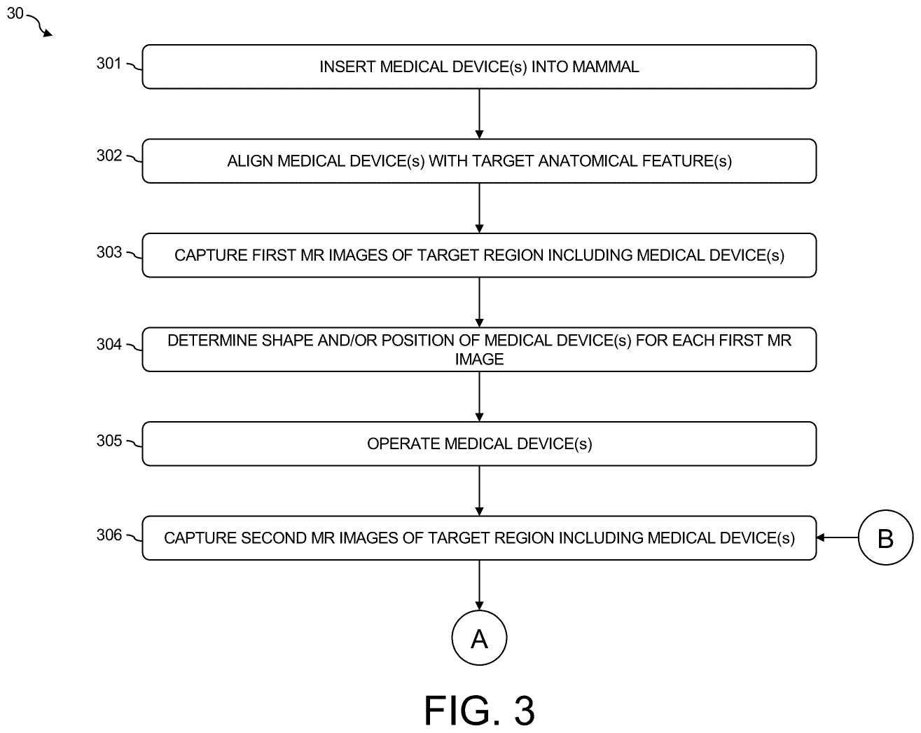

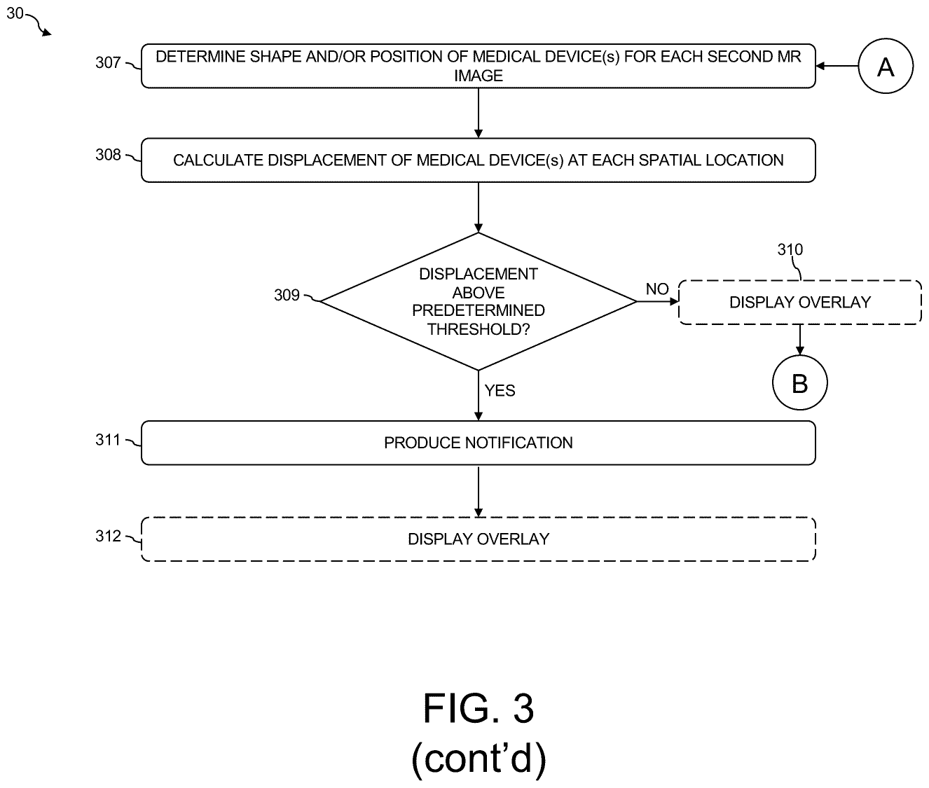

FIG. 3 is a flow chart of a method 30 for controlling delivery of thermal therapy according to one or more embodiments.

In step 301, one or more medical devices is/are inserted into a mammal such as a human. The medical device(s) can comprise a UA 200 and/or an ECD 210. For example, the UA 200 can be inserted through the urethra 202 and between the upper and lower portions of prostate 220. Additionally or alternatively, the ECD 210 can be inserted into the rectum 230.

In step 302, the medical device(s) is aligned with a respective target anatomical feature of the mammal. The medical device(s) can be aligned using MR images. The medical device(s) can include one or more fiducial marks that can be viewable in the MR images to assist with insertion and/or alignment of the respective medical device. For example, the UA 200 (e.g., the ultrasound transducer(s) 208) can be aligned with a target volume of the prostate 220. Additionally or alternatively, the ECD 210 can be aligned with a rectal wall 240 near or adjacent to the prostate 220. An example of an inserted and aligned UA 200 and an inserted and aligned ECD 210 is shown in FIG. 2.

In step 303, first MR images (e.g., a first set of MR images) are captured of a target region of the mammal that includes the inserted and aligned medical device(s) (e.g., the UA and/or the ECD 210). The first MR images are captured in the same MR imaging scan and collection period, sometimes referred to as a dynamic, in which MR images are captured at respective spatial locations along an axis that is orthogonal to the image plane of each MR image. Each first MR image represents a cross-sectional image of one or more target anatomical features and the medical device(s) at a respective spatial location.

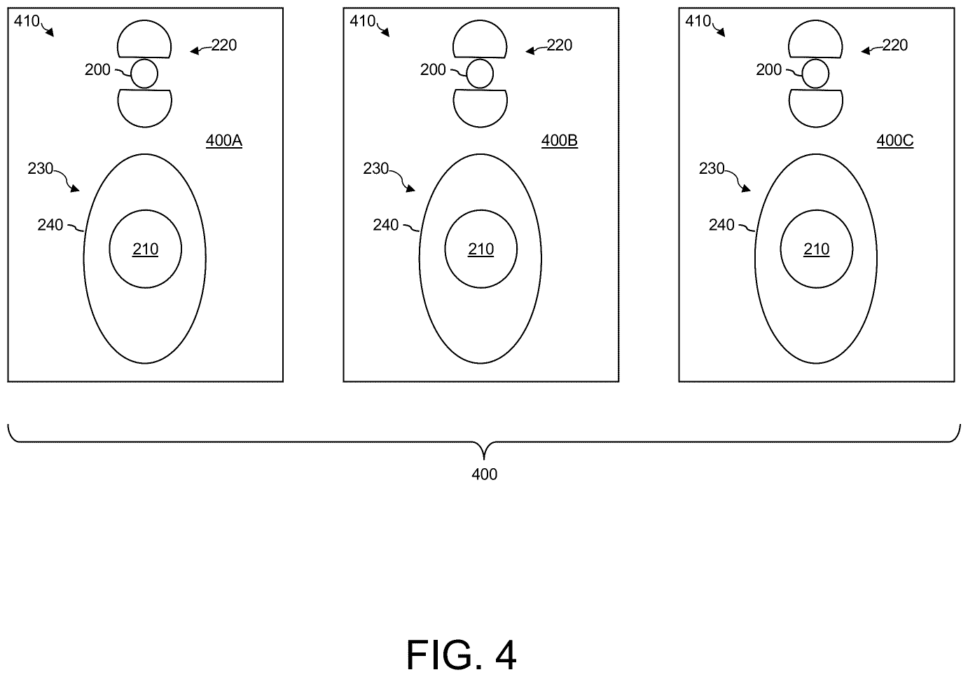

An example set of first MR images 400 is shown in FIG. 4. The example set of first MR images 400 includes first MR images 400A-400C. Each first MR image 400 includes a cross-sectional view of a target region 410 of a mammal that includes the inserted and aligned medical device(s) such as an inserted and aligned UA 200 and/or an inserted and aligned ECD 210. Each first MR image 400 also includes a cross section of at least a portion of one or more target anatomical features such as the rectum 230 (e.g., a rectal wall 240) and/or the prostate 220. Though only 3 first MR images 400A-400C are shown in FIG. 4 for illustrative purposes, it is recognized that the set of first MR images 400 can include 25 to 50 MR images or another number of MR images.

In step 304, a respective shape and/or a respective position of each of the medical device(s) is determined for each first MR image. In one or more embodiments, the respective shape and/or the respective position of each of the medical device(s) can be determined by segmenting each first MR image with one or more trained machine-learning (ML) models running on a computer to determine a boundary, perimeter, or contour of each of the medical device(s). The shape of a medical device can correspond to or be the same as the boundary/perimeter/contour of the medical device. The position of a medical device can correspond to or be the same as a centroid of the medical device's shape and/or to the shape of the medical device. The respective shape and/or the respective position of each of the medical device(s) is determined for the first MR images that were captured over a first time period (e.g., a first dynamic).

In one or more embodiments, a single trained ML model is configured and/or trained to segment each of the medical device(s). In one or more other embodiments, a first trained ML model is configured and/or trained to segment a first medical device (e.g., a UA 200), and a second trained ML model is configured and/or trained to segment a second medical device (e.g., an ECD 210).

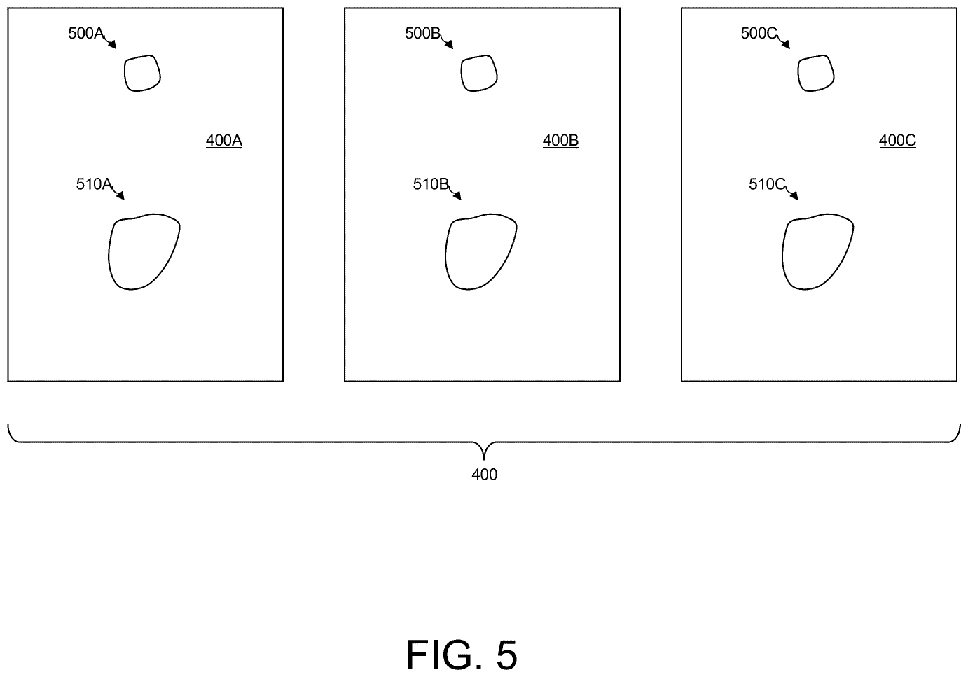

FIG. 5 shows an example shape of each of the medical device(s) for each first MR image 400. A respective cross-sectional shape 500A-500C of the UA 200 at respective spatial locations is determined for each first MR image 400A-400C. Additionally or alternatively, a respective cross-sectional shape 510A-510C of the ECD 210 at respective spatial locations is determined for each first MR image 400A-400C.

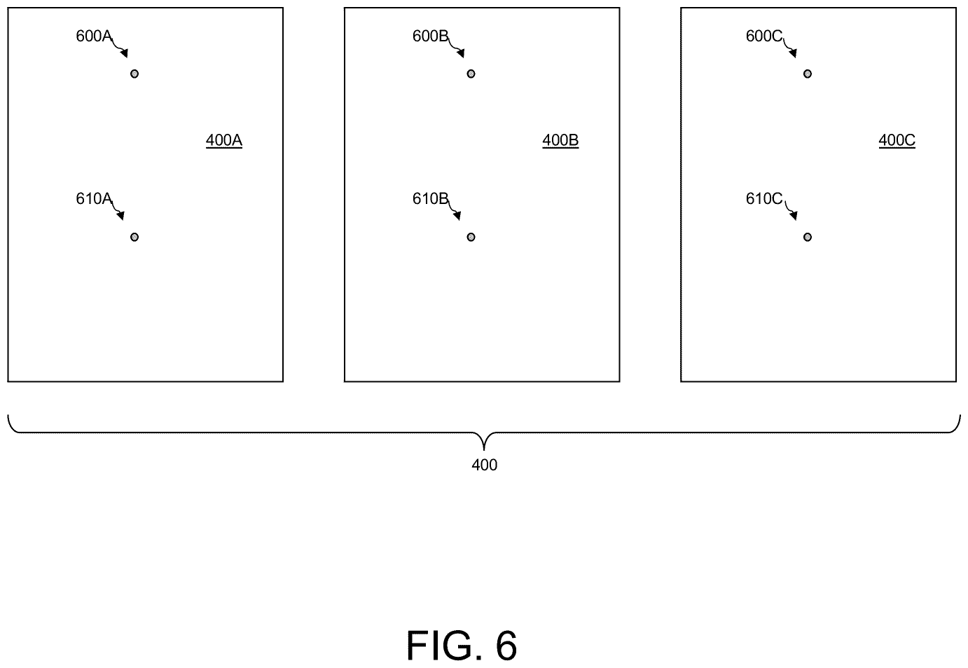

FIG. 6 shows an example centroid of each of the medical device(s) for each first MR image 400. A respective centroid 600A-600C of the UA 200 at respective spatial locations is determined for each first MR image 400A-400C. Additionally or alternatively, a respective centroid 610A-610C of the ECD 210 at respective spatial locations is determined for each first MR image 400A-400C.

In step 305, the medical device(s) is/are operated for example to perform a medical procedure. In one or more embodiments, the medical procedure includes thermal therapy using a UA 200 or another thermal therapy device, for example to perform thermal therapy on the prostate 220. Additionally or alternatively, the medical procedure includes cooling and/or regulating the temperature of the rectum 230 and/or a rectal wall 240 using an ECD 210 or another ECD, for example during a thermal therapy procedure using a UA 200 or another thermal therapy device (e.g., using ultrasound energy, laser energy, and/or other energy).

Steps 306-312 are performed while the medical device(s) is/are operated in step 305.

In step 306, second MR images (e.g., a second set of MR images) are captured of the target region of the mammal that includes the inserted and aligned medical device(s) (e.g., the UA and/or the ECD 210). The second MR images are captured in the same MR imaging scan and collection period (e.g., the same dynamic. Each second MR image represents a cross-sectional image of the same target anatomical feature(s) and the medical device(s) at the same respective spatial location as a respective first MR image.

Step 306 is the same as step 303 except that step 306 is performed later in time than step 303 and that step 306 is performed while the medical device(s) is/are operated in step 305. The example set of first MR images 400 shown in FIG. 4 is representative of the second MR images, though there may be differences in the position and/or shape of the medical device(s) between one or more first MR images 400 and one or more respective second MR images at the same respective spatial location(s).

In step 307 (via placeholder A), a respective shape and/or a respective position of each of the medical device(s) is determined for each second MR image. In one or more embodiments, the respective shape and/or the respective position of each of the medical device(s) can be determined by segmenting each second MR image with one or more trained ML models running on a computer to determine a boundary or perimeter of each of the medical device(s).

Step 307 is the same as step 304 except that step 307 is performed later in time than step 304 and that step 307 is performed while the medical device(s) is/are operated in step 305. The example shapes 500A-500C of the UA 200 and shapes 510A-510C of the ECD 210 shown in FIG. 5 are representative of the shapes of the UA 200 and ECD 210 that can be determined for the second MR images, though there may be differences in one or more shapes of the UA 200 and/or the ECD 210, or the position of one or more shapes of the UA 200 and/or of the ECD 210, between one or more first MR images 400 and one or more respective second MR images at the same respective spatial location(s). The example centroids 600A-600C of the UA 200 and centroids 610A-610C of the ECD 210 shown in FIG. 6 are representative of the centroids of the UA 200 and ECD 210 that can be determined for the second MR images, though there may be differences in the position of one or more centroids of the UA 200 and/or of the ECD 210, between one or more first MR images 400 and one or more respective second MR images at the same respective spatial location(s).

In step 308, the displacement or movement of the each of the medical device(s) is calculated for each spatial location of a respective first MR image and a respective second MR image. In one or more embodiments, the displacement or movement be calculating the distance between the image coordinates of a medical-device centroid determined for each first MR image and the image coordinates of a medical-device centroid determined for a respective second MR image at the same/respective spatial location.

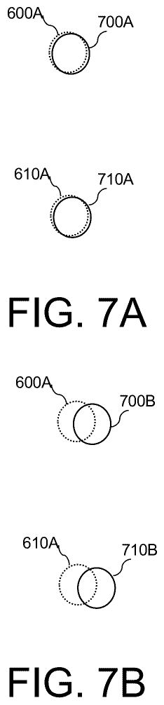

FIG. 7A is a simplified diagram of first centroids 600A, 610A of the UA 200 and ECD 210, respectively, determined from a first MR image corresponding to a first spatial location and second centroids 700A, 710A of the UA 200 and ECD 210, respectively, determined from a second MR image corresponding to the same first spatial location.

FIG. 7B is the same as FIG. 7A except that in FIG. 7B the second centroids 700A, 710A are replaced with second centroids 700B, 710B, respectively, which have a larger displacement relative to the first centroids 600A, 610A, respectively, compared to the second centroids 700A, 710A relative to the first centroids 600A, 610A, respectively.

Though the centroids shown in FIGS. 7A and 7B are shown as large circles for illustrative purposes, in practice the centroids are points having respective coordinates in image space with a known scale. The distance been the first and second centroids 600A, 700A, respectively, of the UA 200 can be calculated using the respective coordinates of the first and second centroids 600A, 700A and the known scale of the first and second MR images, which are captured at the same resolution and scale. The distance been the first and second centroids 610A, 700B, respectively, of the UA 200 can be calculated using the respective coordinates of the first and second centroids 600A, 700B and the known scale of the first and second MR images, which are captured at the same resolution and scale. The distance been the first and second centroids 610A, 710A, respectively, of the ECD 210 can be calculated using the respective coordinates of the first and second centroids 600A, 710A and the known scale of the first and second MR images, which are captured at the same resolution and scale. The distance been the first and second centroids 610A, 710B, respectively, of the ECD 210 can be calculated using the respective coordinates of the first and second centroids 610A, 710B and the known scale of the first and second MR images, which are captured at the same resolution and scale.

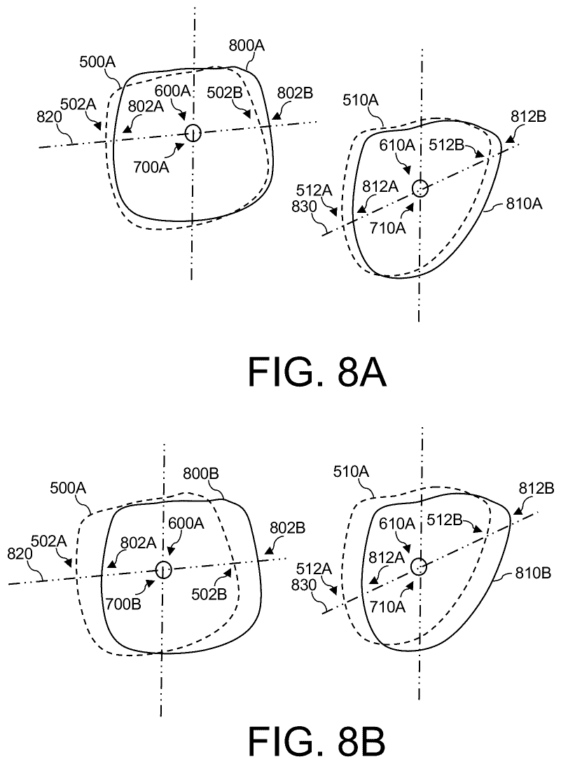

FIG. 8A is a simplified diagram of first shapes 500A, 510A of the UA 200 and ECD 210, respectively, determined from a first MR image corresponding to a first spatial location and second shapes 800A, 810A of the UA 200 and ECD 210, respectively, determined from a second MR image corresponding to the same first spatial location.

The distance been the first and second shapes 500A, 800A, respectively, of the UA 200 can be calculated based on distance between the respective pairs of points 502A, 802A and points 502B, 802B that correspond to the intersection of an axis 820 that passes through the perimeter of the first and second shapes 500A, 800A and the known scale of the first and second MR images, which are captured at the same resolution and scale. Multiple axes 820 in different orientations can be defined through the first and second shapes 500A, 800A to determine the distances between respective pairs of points that correspond to the intersection of a respective axis 820 that passes through the perimeter of the first and second shapes 500A, 800A. One or more statistics can be applied to the calculated distances, for example to determine an average or median distance between the first and second shapes 500A, 800A. In one or more embodiments, each axis 820 can pass through the centroid 600A of the first shape 500A and/or through the centroid 700A of the second shape 800A.

The distance been the first and second shapes 510A, 810A, respectively, of the ECD 210 can be calculated based on distance between the respective pairs of points 512A, 812A and points 512B, 812B that correspond to the intersection of an axis 830 that passes through the perimeter of the first and second shapes 510A, 810A and the known scale of the first and second MR images, which are captured at the same resolution and scale. Multiple axes 830 in different orientations can be defined through the shapes 510A, 810A to determine the distances between respective pairs of points that correspond to the intersection of a respective axis 830 that passes through the perimeter of the first and second shapes 500A, 800A. One or more statistics can be applied to the calculated distances, for example to determine an average or median distance between the first and second shapes 510A, 810A. In one or more embodiments, each axis 830 can pass through the centroid 610A of the first shape 510A and/or through the centroid 710A of the second shape 810A.

FIG. 8B is the same as FIG. 8A except that in FIG. 8B the second shapes 800A, 810A are replaced with second shapes 800B, 810B, respectively, which have a larger displacement relative to the first shapes 500A, 510A, respectively, compared to the second shapes 800A, 810A relative to the first shapes 500A, 510A, respectively.

The distance been the first and second shapes 500A, 800B, respectively, of the UA 200 can be calculated based on distance between the respective pairs of points 502A, 802A and points 502B, 802B that correspond to the intersection of an axis 820 that passes through the perimeter of the first and second shapes 500A, 800B and the known scale of the first and second MR images, which are captured at the same resolution and scale. Multiple axes 820 in different orientations can be defined through the first and second shapes 500A, 800B to determine the distances between respective pairs of points that correspond to the intersection of a respective axis 820 that passes through the perimeter of the first and second shapes 500A, 800B. One or more statistics can be applied to the calculated distances, for example to determine an average or median distance between the first and second shapes 500A, 800B. In one or more embodiments, each axis 820 can pass through the centroid 600A of the first shape 500A and/or through the centroid 700B of the second shape 800B.

The distance been the first and second shapes 510A, 810B, respectively, of the ECD 210 can be calculated based on distance between the respective pairs of points 512A, 812A and points 512B, 812B that correspond to the intersection of an axis 830 that passes through the perimeter of the first and second shapes 510A, 810B and the known scale of the first and second MR images, which are captured at the same resolution and scale. Multiple axes 830 in different orientations can be defined through the shapes 510A, 810B to determine the distances between respective pairs of points that correspond to the intersection of a respective axis 830 that passes through the perimeter of the first and second shapes 500A, 800B. One or more statistics can be applied to the calculated distances, for example to determine an average or median distance between the first and second shapes 510A, 810B. In one or more embodiments, each axis 830 can pass through the centroid 610A of the first shape 510A and/or through the centroid 710B of the second shape 810B.

In step 309, the displacement and/or movement of each of the medical device(s) is compared to a predetermined threshold displacement/movement. If the displacement or movement of each of the medical device(s) is lower than or equal to the predetermined threshold displacement/movement, the method 30 returns to step 306 (via placeholder B) where another set of second MR images is captured (e.g., a next or subsequent dynamic). An example where displacement/movement is lower than or equal to the predetermined threshold is shown in FIG. 7A and in FIG. 8A. In one or more embodiments, a computer can display or overlay the current contour/shape of each medical device on the current MR image(s) (e.g., second/subsequent MR images), such as a magnitude MR image, in optional step 310 after or in parallel with any of steps 306-309.

If the displacement or movement of each of the medical device(s) is higher than the predetermined threshold displacement/movement, the computer produces a notification in step 311. The notification can indicate that the patient (e.g., mammalian) movement is detected and/or that medical device movement is detected. An example where displacement/movement is higher than the predetermined threshold is shown in FIG. 7B and in FIG. 8B. In one or more embodiments, a computer can display or overlay the current contour/shape of each medical device on the current MR image(s) (e.g., second/subsequent MR images), such as a magnitude MR image, in optional step 312 after or in parallel with any of steps 306-309 or step 311.

In one or more embodiments, the first MR images and the second MR images can be sampled such that only a subset of the first and second MR images are used to determine a respective position and/or a respective shape of each medical device(s). The sampling is performed such that a spatial location for each sampled first MR image is the same as the spatial location for each second MR image so that a valid comparison of the position and/or shape of each medical device(s) can be made.

In one or more embodiments, one, some or all steps of method 30 can be performed by or using one or more computers (e.g., a computer 124) and/or one or more controllers. In one or more embodiments, at least steps 303-312 can be performed by or using one or more computers and/or one or more controllers. For example, control signals can be sent by a computer and/or a controller to cause one or more steps to occur, such as capturing MR images (e.g., steps 303, 305), operating one or more medical device(s) (step 305), and processing the MR images in steps 307-312. For example, the shape and/or position of a medical device (e.g., in steps 304 and 307) can be determined using one or more trained ML models running on a computer.

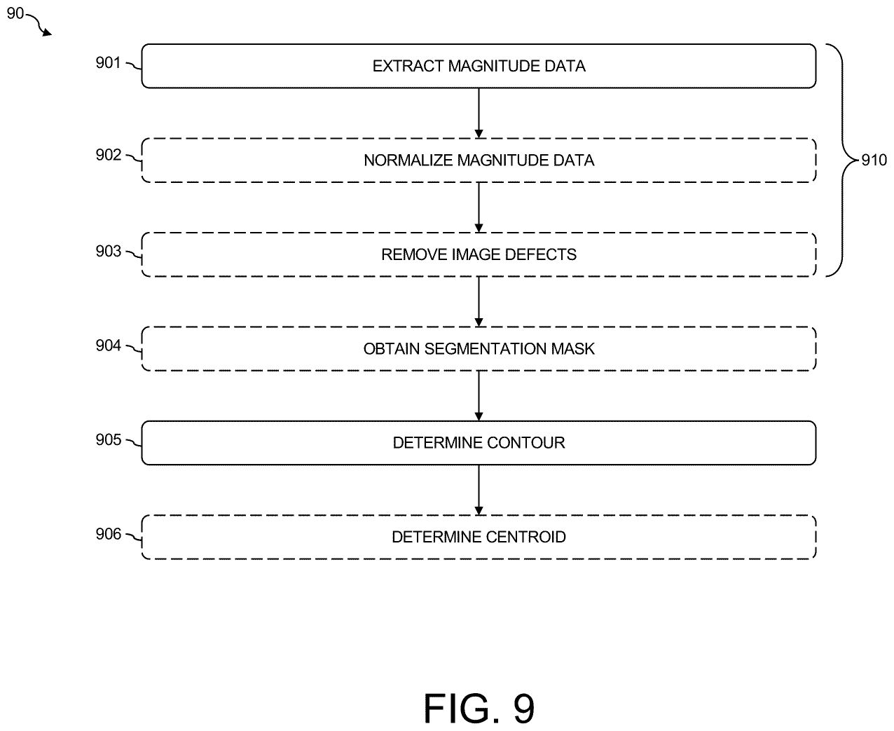

FIG. 9 is a flow chart of a computer-implemented method 90 for determining a respective shape and/or a respective position of a target object in an MR image according to one or more embodiments. The target object can include or can be a medical device, such as a UA 200 or an ECD 210, or an anatomical feature such as the prostate or the rectum. Step 304 and/or step 307 can be performed according to method 90.

In step 901, magnitude data is extracted from the MR image.

In optional step 902, the magnitude data is normalized. For example, the magnitude data can be normalized from 0 to 1. Normalizing the magnitude data can improve the ability of a trained ML model to determine the shape and/or position of the target object.

In optional step 903, image defects such as blobs are removed from the MR image.

In one or more embodiments, a step 910 of preprocessing an MR image includes step 901 and optionally includes step 902 and/or step 903.

In optional step 904, a segmentation mask is obtained for or from a trained ML model that is configured to detect and/or segment the target object.

In step 905, the contour (e.g., shape or perimeter) of the target object is determined using a trained ML model. The contour can be determined using the optional segmentation mask obtained in step 904. Shapes 500A, 500B, 810A, and 810B can represent respective contours of respective medical devices.

In optional step 906, a centroid of the contour is determined and/or calculated. Centroids 600A, 600B, 710A, and 710B are respective contours of respective medical devices.

In one or more embodiments, one, some or all steps of method 90 can be performed by or using one or more computers (e.g., a computer 124) and/or one or more controllers. In one or more embodiments, all steps 901-906 can be performed by or using one or more computers and/or one or more controllers.

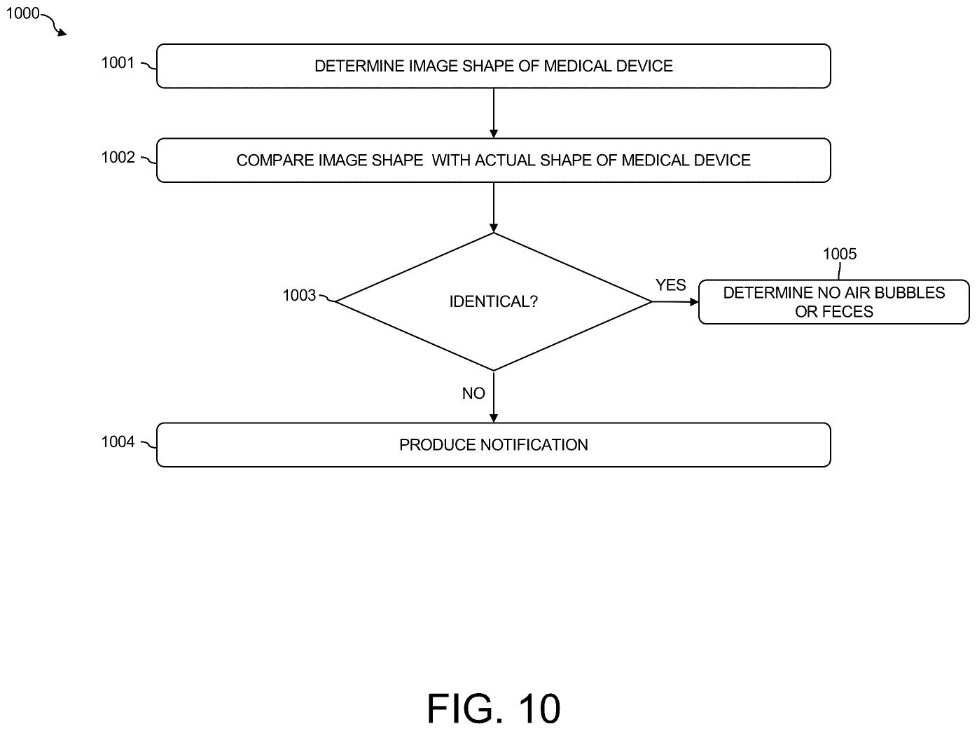

FIG. 10 is a flow chart of a computer-implemented method 1000 for detecting a physical obstruction on a medical device using an MR image according to one or more embodiments.

In step 1001, the image shape or contour of a medical device is determined. The image shape/contour can be determined according to method 90, step 304, or step 307. The medical device can include or can be a UA 200 or a ECD 210.

In step 1002, the image shape/contour is compared with an actual shape (e.g., cross-sectional shape) of the medical device at the same/equivalent spatial location on the medical device corresponding to the spatial location of the MR image.

In step 1003, it is determined whether the image shape/contour and the actual shape are identical or substantially identical.

If the image shape/contour and the actual shape are not identical, a notification or warning is produced in step 1004. The notification/warning can indicate that one or more air bubbles and/or feces is/are detected on the medical device. If the image shape/contour and the actual shape are identical or substantially identical, it is determined in step 1005 that no air bubbles or feces are detected.

In one or more embodiments, method 1000 can be performed for each MR image captured. For example, method 1000 can be performed for each first MR image captured in step 303 and/or for each second MR image captured in step 306.

In one or more embodiments, one, some or all steps of method 1000 can be performed by or using one or more computers (e.g., a computer 124) and/or one or more controllers. In one or more embodiments, all steps 1001-1004 can be performed by or using one or more computers and/or one or more controllers.

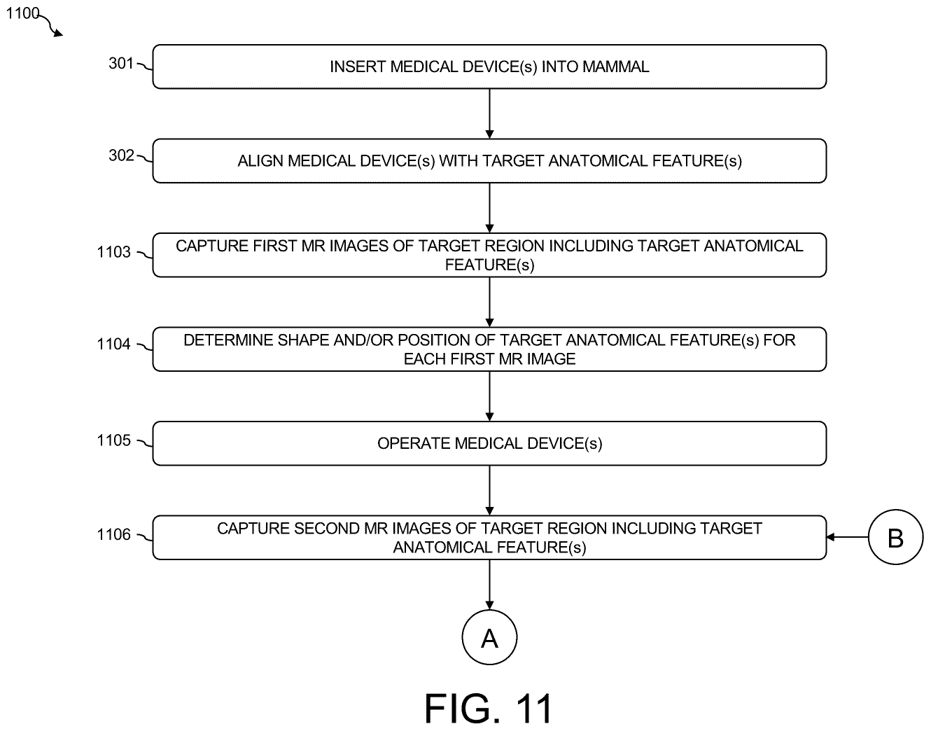

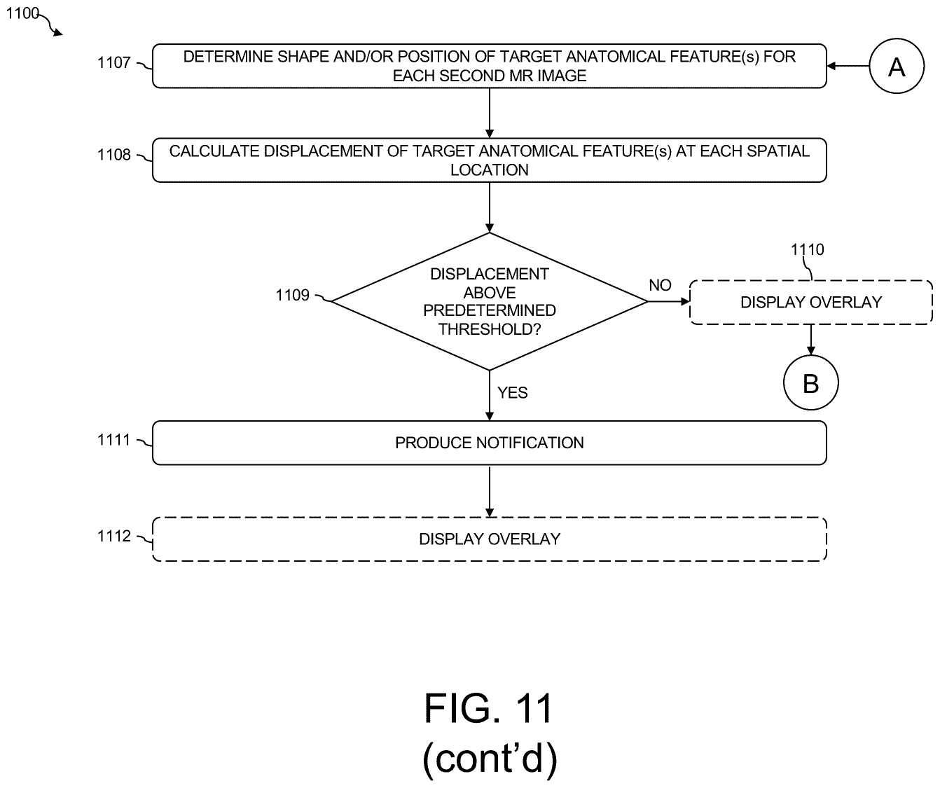

FIG. 11 is a flow chart of a method 1100 for controlling delivery of thermal therapy according to one or more embodiments. Steps 301 and 302 are the same as described with respect to method 30. Steps 1103-1112 are the same as steps 303-312, respectively, except that steps 1103-1112 are performed with respect to one or more target anatomical features. For example, in steps 1104 and 1107 the respective shape and/or the respective position of each target anatomical feature is/are determined in each first MR image and in each second MR image, respectively. The respective shape and/or the respective position of each of the target anatomical feature(s) can be determined by segmenting an MR image (e.g., a first MR image or a second MR image) with one or more trained ML models running on a computer to determine a boundary, perimeter, or contour of each of the target anatomical feature(s). The shape of a target anatomical feature can correspond to or be the same as the boundary/perimeter/contour of the medical device. The position of a target anatomical feature can correspond to or be the same as a centroid of the target anatomical feature's shape and/or to the shape of the target anatomical feature.

In one or more embodiments, a single trained ML model is configured and/or trained to segment each of the target anatomical feature(s). In one or more other embodiments, a first trained ML model is configured and/or trained to segment a target anatomical feature (e.g., a prostate 220), and a second trained ML model is configured and/or trained to segment a second target anatomical feature (e.g., a rectum 230).

In one or more embodiments, a computer can display or overlay the current contour/shape of each of the target anatomical feature(s) on the current MR image(s) (e.g., second/subsequent MR images), such as a magnitude MR image, in optional step 1110 after or in parallel with performing any of steps 1106-1109.

In one or more embodiments, a computer can display or overlay the current contour/shape of each of the target anatomical feature(s) on the current MR image(s) (e.g., second/subsequent MR images), such as a magnitude MR image, in optional step 1112 after or in parallel with performing any of steps 1106-1109 or step 1111.

In one or more embodiments, one, some or all steps of method 1100 can be performed by or using one or more computers (e.g., a computer 124) and/or one or more controllers. In one or more embodiments, at least steps 1103-1112 can be performed by or using one or more computers and/or one or more controllers. For example, control signals can be sent by a computer and/or a controller to cause one or more steps to occur, such as capturing MR images (e.g., steps 1103, 1105), operating one or more medical device(s) (step 1105), and processing the MR images in steps 1107-1112. For example, the shape and/or position of a target anatomical feature (e.g., in steps 1104 and 1107) can be determined using one or more trained ML models running on a computer.

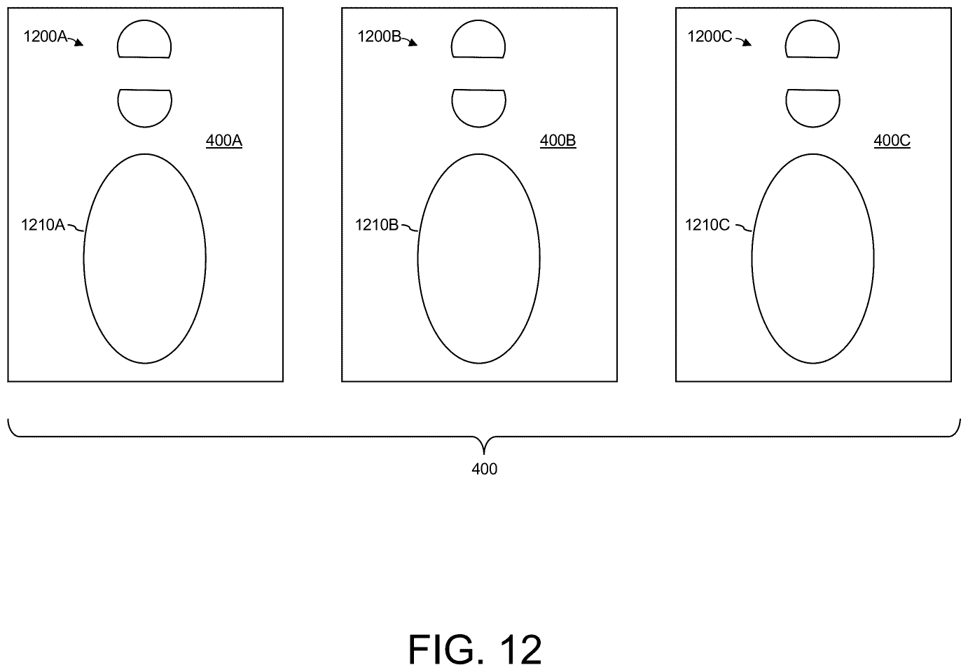

FIG. 12 shows an example shape of each of the target anatomical feature(s) for each first MR image 400. A respective cross-sectional shape 1200A-1200C of the prostate 220 at respective spatial locations is determined for each first MR image 400A-400C. Additionally or alternatively, a respective cross-sectional shape 1210A-1210C of the rectum 230 at respective spatial locations is determined for each first MR image 400A-400C.

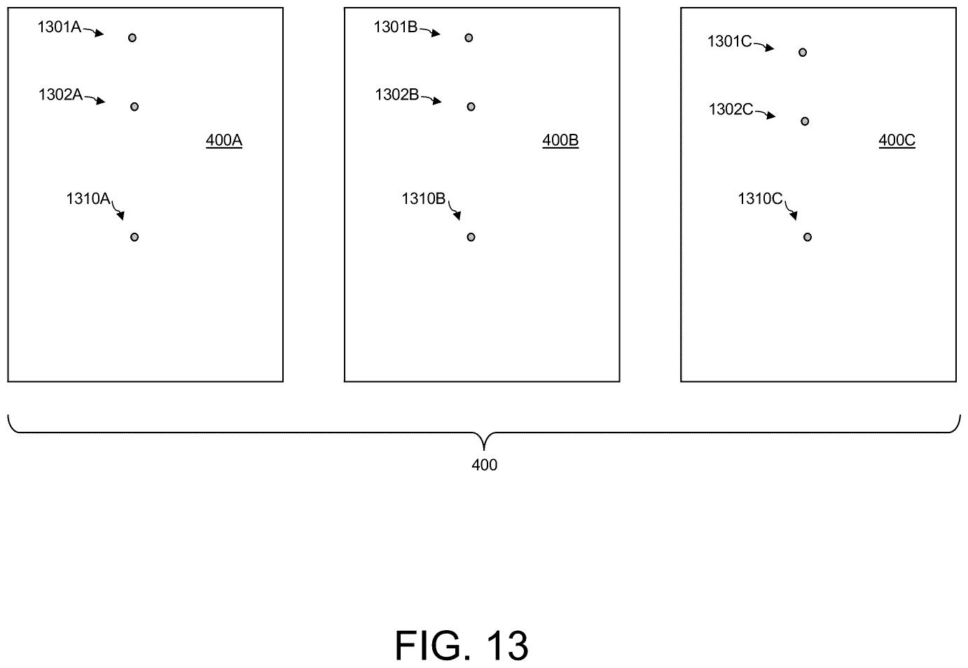

FIG. 13 shows an example centroid(s) of each of the target anatomical feature(s) for each first MR image 400 that can be determined in step 1104. A respective first centroid 1301A-1301C of the upper portion of the prostate 220 and a respective second centroid 1302A-1302C of the lower portion of the prostate 220 at respective spatial locations is determined for each first MR image 400A-400C. Additionally or alternatively, a respective centroid 1310A-1310C of the rectum 230 at respective spatial locations is determined for each first MR image 400A-400C.

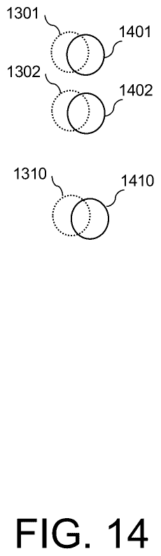

FIG. 14 is a simplified diagram of first centroids 1301, 1302 of the prostate 220 and a first centroid 1310 of the rectum 230 determined from a first MR image corresponding to a first spatial location, and second centroids 1401, 1402 of the prostate 220 and a second centroid 1410 of the rectum 230, determined from a second MR image corresponding to the same first spatial location.

Though the centroids shown in FIG. 14 are shown as large circles for illustrative purposes, in practice the centroids are points having respective coordinates in image space with a known scale. The distance been the first and second centroids 1301, 1401 of the upper portion of the prostate 220 can be calculated using the respective coordinates of the first and second centroids 1301, 1401 and the known scale of the first and second MR images, which are captured at the same resolution and scale. The distance been the first and second centroids 1302, 1402 of the lower portion of the prostate 220 respectively can be calculated using the respective coordinates of the first and second centroids 1302, 1402 and the known scale of the first and second MR images, which are captured at the same resolution and scale. The distance been the first and second centroids 1310, 1410, respectively, of the rectum 230 can be calculated using the respective coordinates of the first and second centroids 1310, 1410 and the known scale of the first and second MR images, which are captured at the same resolution and scale.

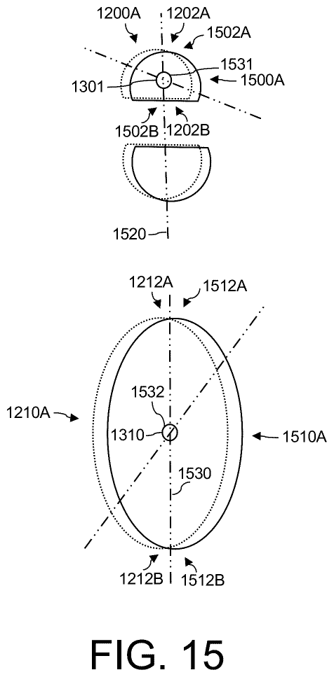

FIG. 15 is a simplified diagram of first shapes 1200A, 1210A of the prostate 220 and the rectum 230, respectively, determined from a first MR image corresponding to a first spatial location and second shapes 1500A, 1510A of the prostate 220 and the rectum 230, respectively, determined from a second MR image corresponding to the same first spatial location.

The distance been the first and second shapes 1200A, 1500A, respectively, of the UA 200 can be calculated based on distance between the respective pairs of points 1202A, 1502A and points 1202B, 1502B that correspond to the intersection of an axis 1520 that passes through the perimeter of the first and second shapes 1200A, 1500A (e.g., of the upper (or lower) portion of the prostate 220) and the known scale of the first and second MR images, which are captured at the same resolution and scale. Multiple axes 1520 in different orientations can be defined through the first and second shapes 1200A, 1500A to determine the distances between respective pairs of points that correspond to the intersection of a respective axis 1520 that passes through the perimeter of the first and second shapes 1200A, 1500A (e.g., of the upper (or lower) portion of the prostate 220). One or more statistics can be applied to the calculated distances, for example to determine an average or median distance between the first and second shapes 1200A, 1500A. In one or more embodiments, each axis 1520 can pass through a centroid 1301 of the first shape 1200A and/or through a centroid 1531 of the second shape 1500A.

The distance been the first and second shapes 1210A, 1510A, respectively, of the rectum 230 can be calculated based on distance between the respective pairs of points 1212A, 1512A and points 1212B, 1512B that correspond to the intersection of an axis 1530 that passes through the perimeter of the first and second shapes 1210A, 1510A and the known scale of the first and second MR images, which are captured at the same resolution and scale. Multiple axes 1530 in different orientations can be defined through the shapes 1210A, 1510A to determine the distances between respective pairs of points that correspond to the intersection of a respective axis 1530 that passes through the perimeter of the first and second shapes 1210A, 1510A. One or more statistics can be applied to the calculated distances, for example to determine an average or median distance between the first and second shapes 1210A, 1510A. In one or more embodiments, each axis 1530 can pass through a centroid 1310 of the first shape 510A and/or through a centroid 1532 of the second shape 1510A.

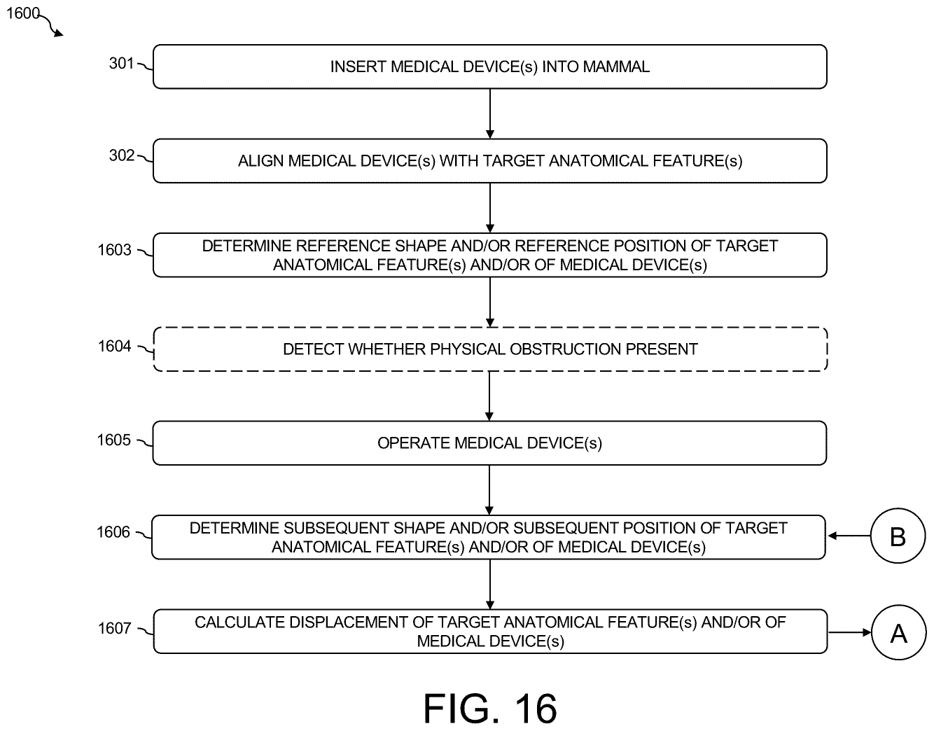

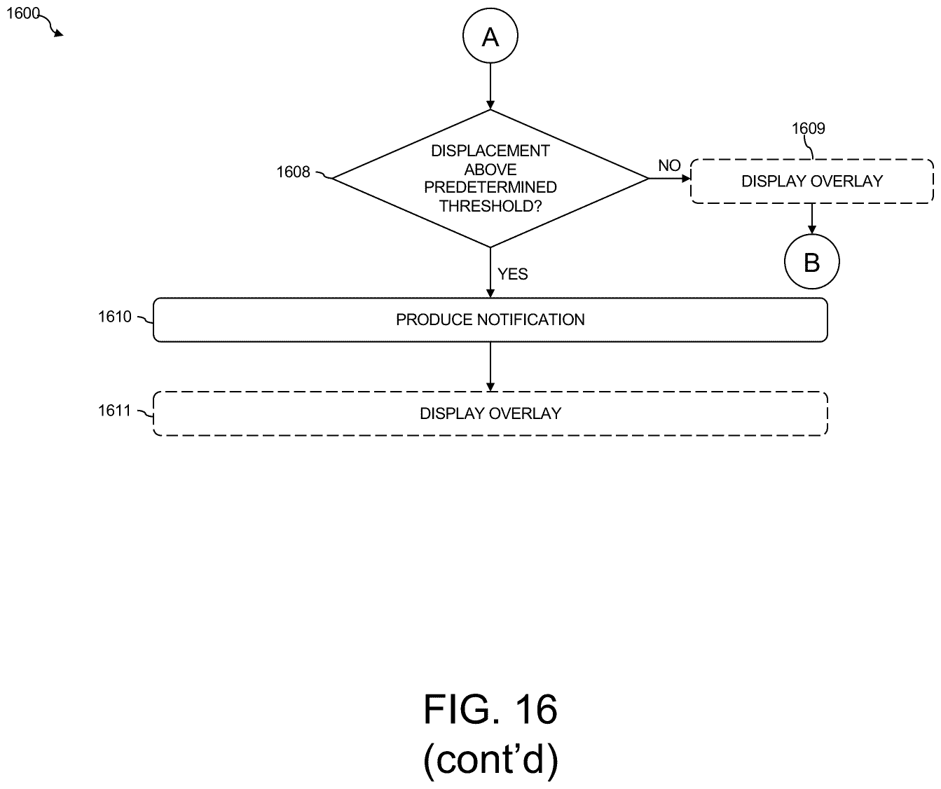

FIG. 16 is a flow chart of a method 1600 for controlling delivery of a medical procedure, such as thermal therapy, according to one or more embodiments.

Steps 301 and 302 are the same as described herein.

In step 1603, a reference shape and/or a reference position of one or more target objects is/are determined. The target object can include one or more target medical devices (e.g., a UA 200, an ECD 210, and/or another target medical device) and/or one or more target anatomical features (e.g., the prostate 220, the rectum 230, and/or another anatomical feature). When the target object(s) includes one or more target medical devices, step 1603 can be performed according to steps 303 and 304. When the target object(s) includes one or more target anatomical features, step 1603 can be performed according to steps 1103 and 1104. It is noted that the same initial/first MR images can be used to perform step 1603 when the target object(s) include both one or more target medical devices and one or more target anatomical features. For example, either step 303 or step 1103 can be performed to capture the initial/first MR images provided that the initial/first MR images show the one or more target medical devices and the one or more target anatomical features of interest.

In optional step 1604, the system can detect whether a physical obstruction is present on or proximal to a surface of one or more medical device(s). Step 1604 can be performed according to method 1000.

Step 1605 can be the same as step 305 and/or step 1105.

Steps 1606-1609 are performed while the medical device(s) is/are operated in step 1605.

In step 1606, a subsequent shape and/or a subsequent position of the one or more target objects is/are determined. When the target object(s) includes one or more target medical devices, step 1606 can be performed according to steps 306 and 307. When the target object(s) includes one or more target anatomical features, step 1606 can be performed according to steps 1106 and 1107. It is noted that the same subsequent/second MR images can be used to perform step 1606 when the target object(s) include both one or more target medical devices and one or more target anatomical features. For example, either step 306 or step 1106 can be performed to capture the subsequent/second MR images provided that the subsequent/second MR images show the one or more target medical devices and the one or more target anatomical features of interest.

In step 1607, a displacement and/or movement of the of the one or more target objects is/are determined. When the target object(s) includes one or more target medical devices, step 1607 can be performed according to step 308. When the target object(s) includes one or more target anatomical features, step 1607 can be performed according to step 1108.

In step 1608 (via placeholder A), the displacement and/or movement of each of the one or more target objects is compared to a predetermined threshold displacement/movement. The predetermined threshold displacement/movement can be the same for each of the one or more target objects in one or more embodiments. In one or more other embodiments, a first predetermined threshold displacement/movement can be used for each target medical device and a second predetermined threshold displacement/movement can be used for each target anatomical feature. In one or more other embodiments, a different displacement/movement can be used for each target object (e.g., each target medical device and/or each target anatomical feature).

When the target object(s) includes one or more target medical devices, step 1608 can be performed according to step 309. When the target object(s) includes one or more target anatomical features, step 1608 can be performed according to step 1109

When the displacement and/or movement of at least one of the one or more target objects is higher than a predetermined threshold displacement/movement (i.e., step 1608=yes), a notification is produced in step 1609. Step 1609 can be the same as step 310 and/or step 1110. In one or more other embodiments, when the displacement and/or movement of at least a predetermined number of the one of the one or more target objects is higher than a predetermined threshold displacement/movement (i.e., step 1608=yes), a notification is produced in step 1609.

When the displacement and/or movement of all of the one or more target objects is lower than or equal to a predetermined threshold displacement/movement (i.e., step 1608=no), the method 1600 returns to step 1604 (via placeholder B) to determine a subsequent shape and/or a subsequent position of the one or more target objects at a next/subsequent time period. In one or more other embodiments, when the displacement and/or movement of at least a predetermined number of the one of the one or more target objects lower than or equal to a predetermined threshold displacement/movement (i.e., step 1608=no), the method 1600 returns to step 1604 (via placeholder B).

In one or more embodiments, a computer can display or overlay the current contour/shape of each of the target objects(s) on the current MR image(s) (e.g., second/subsequent MR images), such as a magnitude MR image, in optional step 1609, for example after or in parallel with one or more of steps performing step 1606-1608.

In one or more embodiments, a computer can display or overlay the current contour/shape of each of the target anatomical feature(s) on the current MR image(s) (e.g., second/subsequent MR images), such as a magnitude MR image, in optional step 1611, for example after or in parallel with one or more of steps performing step 1606-1608 or step 1610.

In one or more embodiments, one, some or all steps of method 1600 can be performed by or using one or more computers (e.g., a computer 124) and/or one or more controllers. In one or more embodiments, at least steps 1603-1609 can be performed by or using one or more computers and/or one or more controllers. For example, control signals can be sent by a computer and/or a controller to cause one or more steps to occur, such operating one or more medical device(s) (step 1605), and processing the MR images in steps 1603 and steps 1607-1609. For example, the shape and/or position of a target object (e.g., in steps 1603 and 1606) can be determined using one or more trained ML models running on a computer.

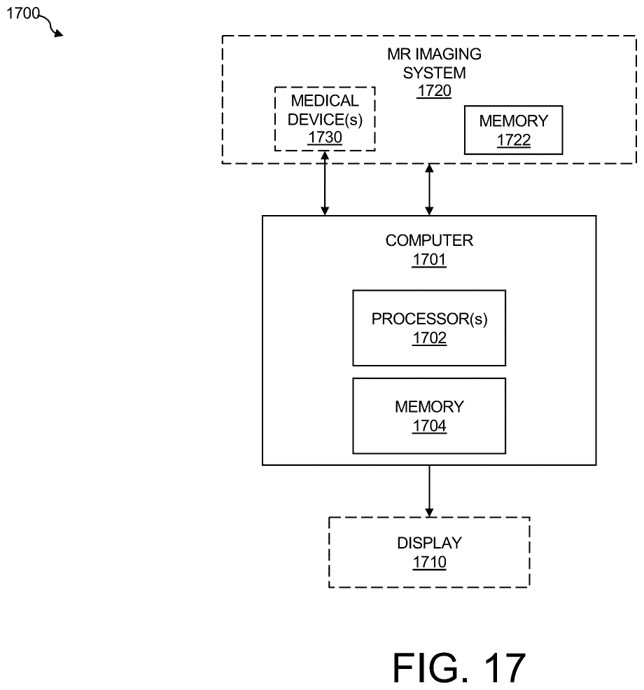

FIG. 17 is a block diagram of a system 1700 according to one or more embodiments. The system 1700 includes at least a computer 1701 that can be configured to perform one or more tasks, one or more steps, and/or one or more methods as described herein. The computer 1701 includes one or more processors 1702 (e.g., one or more microprocessors and/or other hardware-based processors) and computer memory 1704 in communication with and/or operably coupled to the processor(s) 1702. The computer memory 1704 includes at least non-volatile computer memory that stores computer-readable instructions that are can be executed by the processor(s) 1702 to perform one or more tasks, one or more steps, and/or one or more methods as described herein.

The computer 1701 can be in communication with an optional display 1710, such as a display screen. The computer 1701 can cause the display 1710 to display MR images and/or overlays of (a) the position(s) and/or shape(s) of target object(s) and (b) one or more MR images that includes the target object(s). In one or more embodiments, the system 1700 includes the optional display 1710.

The computer 1701 can be in communication with an optional MR imaging system 1720. The computer 1701 generate control signals that can cause the MR imaging system 1720 to capture MR images of a mammal, for example at first and second times (or first and second timeframes). Additionally or alternatively, the computer 1701 can receive MR images of a mammal (e.g., captured at first and second times/timeframes) from the MR imaging system 1720, for example from computer memory 1722 (e.g., non-volatile computer memory) on/in the MR imaging system 1720. The MR imaging system 1720 can be the same as the MR system 102 (FIG. 1). In one or more embodiments, the system 1700 includes the optional MR imaging system 1720 and/or the optional memory 1722.

The computer 1701 can be in communication with one or more medical devices 1730 that can be inserted into a mammal during a medical procedure, such as a thermal therapy procedure. The medical device(s) 1730 can be the same as a UA 200 and/or an ECD 210 (FIG. 2). The medical devices 1730 and a mammal can be imaged by the MR imaging system 1720 prior to and during the medical procedure. The computer 1701 or another computer can produce control signals that cause at least one of the medical device(s) 1730 to start, stop, and/or perform a medical procedure such as a thermal therapy procedure. In one or more embodiments, the system 1700 includes one or more of the medical device(s) 1730.

In one or more embodiments, the system 1700 can be configured to perform one, some, or all steps of method 30, of method 90, of method 1000, of method 1100, and/or of method 1600.

The invention should not be considered limited to the particular embodiments described above. Various modifications, equivalent processes, as well as numerous structures to which the invention may be applicable, will be readily apparent to those skilled in the art to which the invention is directed upon review of this disclosure. The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of this application need not reside on a single computer or processor but may be distributed in a modular fashion among a number of different computers or processors to implement various aspects of this application.

Computer-executable instructions may be in many forms, such as program modules, executed by one or more computers or other devices. Generally, program modules include routines, programs, objects, components, data structures, etc. that performs particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the disclosure and claims include new and novel improvements to existing methods and technologies, which were not previously known nor implemented to achieve the useful results described above. Users of the method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A computer configured to monitor motion during a medical procedure, comprising:
one or more processors; and
non-volatile computer-readable memory operably coupled to the one or more processors, the non-volatile computer-readable memory storing computer-readable instructions that, when executed by the one or more processors, cause the one or more processors to:
receive reference magnetic resonance (MR) images of a target volume of a mammal, the target volume including one or more target objects, the reference MR images captured over a first time period;
determine a reference shape of each target object from one or more of the reference MR images using one or more trained machine-learning (ML) models running on the computer, each reference MR image captured at a respective spatial location in the target volume;
receive subsequent MR images of the target volume, the subsequent MR images captured over a second time period that occurs after the first time period;
determine a subsequent shape of each target object from one or more of the subsequent MR images using the one or more trained ML models, each subsequent MR image captured at the same respective spatial location in the target volume as a corresponding reference MR image;
compare each subsequent shape for each target object to a corresponding reference shape for a corresponding target object, wherein a comparison of a given subsequent shape and a given reference shape is performed using a corresponding subsequent MR image and a corresponding reference MR image that were captured at the same respective spatial location in the target volume;
calculate a respective movement of each target object based, at least in part, on the comparison; and
when the respective movement is greater than a predetermined threshold, produce a movement notification.

2. The computer system of claim 1, wherein the one or more target objects includes one or more target anatomical features of the mammal.

3. The computer system of claim 2, wherein the one or more target anatomical features includes a prostate.

4. The computer system of claim 1, wherein the one or more target objects includes one or more medical devices.

5. The computer system of claim 4, wherein the one or more medical devices includes a thermal therapy applicator and/or an endorectal cooling device.

6. The computer system of claim 5, wherein the computer-readable instructions, when executed by the one or more processors, further cause the one or more processors to cause the thermal therapy applicator to start a thermal therapy procedure after determining the reference shape of each target object.

7. The computer system of claim 5, wherein the computer-readable instructions, when executed by the one or more processors, further cause the one or more processors to:
compare the reference shape of the endorectal cooling device with a known shape of the of the endorectal cooling device; and
produce a physical obstruction notification when the reference shape is different than the known shape.

8. The computer system of claim 1, wherein the computer-readable instructions, when executed by the one or more processors, further cause the one or more processors to:
calculate a reference centroid of each reference shape;
calculate a subsequent centroid of each subsequent shape;
determine a respective distance between a position of each subsequent centroid for each subsequent shape to a position of a corresponding reference centroid for the corresponding reference shape; and
calculate the respective movement of each target object based, at least in part, on the respective distance.

9. The computer system of claim 1, wherein the first time period occurs before a start of the medical procedure and the second time period occurs during the medical procedure.

10. A method for controlling a delivery of thermal therapy, comprising:
inserting a thermal therapy applicator into a mammal;
capturing first magnetic resonance (MR) images, with an MR imaging system, of the mammal at a first time, the first MR images representing first cross-sectional images of the mammal including an inserted thermal therapy applicator, the first cross-sectional images at respective spatial locations in the mammal;
segmenting the first MR images with a trained machine-learning (ML) model running on the computer, the trained ML model having been trained with reference segmented MR images that include a reference thermal therapy applicator;
determining, with the computer, a respective first shape and/or a respective first position of the inserted thermal therapy applicator at each spatial location;
applying thermal therapy, with the inserted thermal therapy applicator, to a target volume in the mammal; and
while applying the thermal therapy:
a. capturing second MR images, with the MR imaging system, of the mammal at a second time, the second MR images representing second cross-sectional images of the mammal and the inserted thermal therapy applicator, the second cross-sectional images at the respective spatial locations;
b. segmenting the second MR images with the trained ML model;
c. determining, with the computer, a respective second shape and/or a respective second position of the inserted thermal therapy applicator at each spatial location;
d. calculating, with the computer, a displacement of the inserted thermal therapy applicator at each spatial location by comparing the respective first and second shapes and/or the respective first and second positions of the inserted thermal therapy applicator at each spatial location; and
e. producing a notification, with the computer, when the displacement of the inserted thermal therapy applicator is greater than a predetermined threshold value.

11. The method of claim 10, further comprising:
preprocessing the first MR images, wherein segmenting the first MR images comprises segmenting first preprocessed MR images; and
preprocessing the second MR images, wherein segmenting the second MR images comprises segmenting second preprocessed MR images.

12. The method of claim 11, wherein:
preprocessing the first MR images includes:
extracting magnitude data for each first MR image; and
normalizing the magnitude data for each first MR image; and
preprocessing the second MR images includes:
extracting magnitude data for each second MR image; and
normalizing the magnitude data for each second MR image.

13. The method of claim 10, further comprising displaying, on a display screen in communication with the computer, an overlay of (a) one or more of the second MR images and (b) the respective second shape and/or the respective second position of the inserted thermal therapy applicator corresponding to the one or more of the second MR images.

14. The method of claim 10, further comprising:
determining, with the computer, a respective first centroid of the respective first shape; and
determining, with the computer, a respective second centroid of the respective second shape,
wherein the respective displacement is calculated using the respective first and second centroids corresponding to the respective spatial location.

15. The method of claim 10, further comprising repeating steps a-e in a loop while applying the thermal therapy.

16. A system for controlling a delivery of thermal therapy, comprising:
a magnetic resonance (MR) imaging system;
a computer in communication with the MR imaging system, the computer including:
one or more processors; and
non-volatile computer-readable memory operably coupled to the one or more processors, the non-volatile computer-readable memory storing computer-readable instructions that, when executed by the one or more processors, cause the one or more processors to:
receive first MR images of a mammal at a first time, the first MR images representing first cross-sectional images of the mammal including an inserted thermal therapy applicator, the first cross-sectional images at respective spatial locations in the mammal;
segment the first MR images with a trained machine-learning (ML) model running on the computer, the trained ML model having been trained with reference segmented MR images that include a reference thermal therapy applicator;
determine a respective first shape and/or a respective first position of the inserted thermal therapy applicator at each spatial location;
while the thermal therapy is applied with the inserted thermal therapy applicator:
a. receive second MR images of the mammal at a second time, the second MR images representing second cross-sectional images of the mammal and the inserted thermal therapy applicator, the second cross-sectional images at the respective spatial locations;
b. segment the second MR images with the trained ML model;
c. determine a respective second shape and/or a respective second position of the inserted thermal therapy applicator at each spatial location;
d. calculate a displacement of the inserted thermal therapy applicator at each spatial location by comparing the respective first and second shapes and/or the respective first and second positions of the inserted thermal therapy applicator at each spatial location; and
e. produce a notification when the displacement of the inserted thermal therapy applicator is greater than a predetermined threshold value.

17. The system of claim 16, wherein the computer-readable instructions, when executed by the one or more processors, further cause the one or more processors to display, on a display screen in communication with the computer, an overlay of (a) one or more of the second MR images and (b) the respective second shape and/or the respective second position of the inserted thermal therapy applicator corresponding to the one or more of the second MR images.

18. The system of claim 16, wherein the computer-readable instructions, when executed by the one or more processors, further cause the one or more processors to:
determine a respective first centroid of the respective first shape; and
determine a respective second centroid of the respective second shape,
wherein the respective displacement is calculated using the respective first and second centroids corresponding to the respective spatial location.

19. The system of claim 16, wherein the computer-readable instructions, when executed by the one or more processors, further cause the one or more processors to repeat steps a-e in a loop the thermal therapy is applied with the inserted thermal therapy applicator.

20. The system of claim 16, wherein the computer-readable instructions, when executed by the one or more processors, further cause the one or more processors to:
preprocess the first MR images, wherein segmenting the first MR images comprises segmenting first preprocessed MR images; and
preprocess the second MR images, wherein segmenting the second MR images comprises segmenting second preprocessed MR images.
